(19) United States
(12) Patent Application Publication
(10) Pub. No.: US 2022/0267433 A1
(43) Pub. Date: Aug. 25, 2022

(54) USE OF IL-1ß BINDING ANTIBODIES FOR TREATING NEUROINFLAMMATORY DISORDERS

(71) Applicant: Novartis AG, Basel (CH)

(72) Inventors: Jang-Ho Cha, Arlington, MA (US); Kristin Hannesdottir, Boston, MA (US)

(21) Appl. No.: 17/649,867

(22) Filed: Feb. 3, 2022

Related U.S. Application Data
(60) Provisional application No. 63/145,135, filed on Feb. 3, 2021.

(51) Int. Cl. C07K 16/24; A61P 25/28

(52) U.S. Cl. CPC C07K 16/245; A61P 25/28; A61K 2039/505

(57) ABSTRACT

Use of an IL-1β binding antibody or a functional fragment thereof, especially canakinumab or a functional fragment thereof, or gevokizumab or a functional fragment thereof, dosing regimen and biomarkers for the treatment of neuroinflammatory disorders, e.g., Alzheimer's disease (AD).

### CROSS REFERENCE TO RELATED APPLICATIONS

This application claims the benefit of of U.S. Provisional Application No. 63/145,135, filed Feb. 3, 2021, the contents of the aforementioned application are hereby incorporated by reference in its entirety.

### SEQUENCE LISTING

The instant application contains a Sequence Listing which has been submitted electronically in ASCII format and is hereby incorporated by reference in its entirety. Said ASCII copy, created on Feb. 3, 2022, is named PAT059028-US-NP_SL.txt and is 9,101 bytes in size.

### TECHNICAL FIELD

The present application relates to the use of an IL-1β binding antibody or a functional fragment thereof for the treatment of neuroinflammatory disorders, e.g., Alzheimer's disease (AD).

### BACKGROUND

Alzheimer's disease (AD) is a progressive neurodegenerative disorder characterized by progressive loss of cognitive function and independence and remains one of the highest unmet medical and societal needs. With a very high trial failure rate and to date only one FDA approved treatment (aducanumab) tackling amyloid plaques in the brain of patients suffering from Alzheimer's disease, there is still a need of drug development in AD as well as more innovative trial designs, endpoints, novel biomarkers, and therapeutic targets and treatments to slow, stop or prevent the disease.

AD pathology is characterized by extracellular plaques containing aggregated amyloid-β (Aβ) peptides and intracellular neurofibrillary tangles (NFTs) containing hyperphosphorylated and aggregated tau. Accumulating evidence points towards neuroinflammation as a key mechanism in the pathogenesis of AD. Neuroinflammation, or inflammation of the nervous system, involves inflammatory cytokines; and microglial and astrocyte activation. Pro-inflammatory cytokines are important not only in inflammatory responses, but also in neurogenesis and neuroprotection. However, sustained release of pro-inflammatory cytokines leads to chronic neuroinflammation, which may contribute to a variety of medical conditions including AD. In AD, deposition of Aβ peptides initiates cerebral neuroinflammation mediated by activated microglia. Activated microglia may play a potentially detrimental role in AD by eliciting the expression of pro-inflammatory cytokines such as interleukin (IL)-1β, IL-6, and tumor necrosis factor-α (TNF-α).

Evidence from both in vitro and in vivo models support the notion that misfolded and aggregated proteins trigger an innate immune response and increase the levels of inflammatory mediators in the brain, which contribute to disease severity and progression. Several genes involved in glial clearance of misfolded proteins and inflammatory reaction have been associated with increased risk of AD. It has been shown that in the absence of neuroinflammation, clinical symptoms of dementia do not occur in some individuals with high amyloid plaque load and high Braak stages of neurofibrillary tangles (Perez-Nievas et al 2013). Moreover, Apolipoprotein E4 (apoE4; the most prevalent genetic risk factor of AD) positive individuals with chronic low-grade inflammation have been found to be at a significantly higher risk of AD and an earlier disease onset compared with ApoE4 carriers without chronic inflammation (Tao et al 2018). Numerous studies suggest that chronic systemic inflammatory conditions such as periodontitis (Teixeira et al 2017), rheumatoid arthritis (Chou et al 2016), and sleep apnea (Polsek et al 2018) are likely to affect the immunological processes of the brain and further promote AD progression. In addition, an anti-inflammatory anti-TNF drug, etanercept, was associated with lowered risk of AD among rheumatoid arthritis participants (Chou et al 2016). Finally, asthma drugs have been shown to reduce levels of amyloid beta (AB) in rodent models (Hori et al 2015), as well as modulate inflammation in the brain (microglia activation) and cell death (apoptosis; Zhang et al 2018; Wang et al 2014).

Although recent efforts have largely focused on discovering disease-modifying treatments, there continues to be a high-unmet need for improved symptomatic treatment of AD.

Equally, there is a need to treat neurodegerative disorders of inflammatory origin, namely neuroinflammatory disorders.

### SUMMARY

Provided herein are uses and methods of use of an IL-1β binding antibody or a functional fragment thereof for the treatment of neuroinflammatory disorders or diseases.

Provided herein are uses and methods of use of an IL-1β binding antibody or a functional fragment thereof for the treatment of Alzheimer's disease (AD).

Provided herein is a therapy for the treatment of AD. Provided herein is a novel use of an IL-1β binding antibody or a functional fragment thereof, e.g., canakinumab or a functional fragment thereof, for the treatment of AD.

In particular, provided herein is use of a novel dosing regimen of an anti-IL-1β antagonist, e.g., canakinumab (ACZ885 or Ilaris®) in patients with early AD, i.e., patients with mild cognitive impairment (MCI) due to AD and mild AD.

In another aspect, provided herein is a particular clinical dosage regimen for the administration of an IL-1β binding antibody or a functional fragment thereof, e.g., canakinumab or a functional fragment thereof, for the treatment of AD.

Also provided are methods of treating, in a human subject in need thereof, the methods comprising administering to the subject a therapeutically effective amount of an IL-1β binding antibody or a functional fragment thereof.

Also provided herein is the use of an IL-1β binding antibody or a functional fragment thereof, for the manufacture of a medicament for the treatment of Alzheimer's disease.

Also provided herein is a pharmaceutical composition comprising a therapeutically effective amount of an IL-1β binding antibody (e.g., canakinumab) or a functional fragment thereof, for the treatment of Alzheimer's disease. In certain aspects, the IL-1β binding antibody or a functional fragment thereof is administered at a dose equal or more than 30 mg per treatment. In one aspect, the IL-1β binding antibody or a functional fragment thereof is canakinumab, and is administered at a dose of about 150 mg to about 300 mg per treatment, or at least 150 mg per treatment, or 300 mg per treatment, or at a dose of 150 mg to about 300 mg per treatment. In another aspect, the IL-1β binding antibody or a functional fragment thereof is gevokizumab or a functional fragment thereof, and is administered at a dose about 30 mg to 180 mg per treatment, or about 60 mg to 120 mg per treatment. Such administration can be, e.g., every two weeks, every three weeks, or every four weeks (monthly); and can be administered subcutaneously, or intravenously, and/or in a liquid form contained in a prefilled syringe or as a lyophilized form for reconstitution. Such administration can be, e.g., every four weeks (monthly); and can be administered subcutaneously, or intravenously, and/or in a liquid form contained in a prefilled syringe or as a lyophilized form for reconstitution.

Provided herein are also uses and methods of using serum IL-6 level as a biomarker in the diagnosis of neuroinflammatory disorders, e.g., AD, as well as patient selection for treatment of a neuroinflammatory disorder, e.g., AD. Also provided herein are uses and methods of using serum IL-6 level as a biomarker in the treatment a neuroinflammatory disorder, including AD, in a patient.

Also provided herein is serum IL-6 level for use as a biomarker in the diagnosis of neuroinflammatory disorders, e.g., AD, as well as for use as patient selection for treatment of a neuroinflammatory disorder, In a further aspect provided herein is a patient's serum IL-6 level as a biomarker in the treatment of neuroinflammation, e.g., AD, in a patient, wherein said patient is treated with an IL-1β binding antibody or a functional fragment thereof, e.g., gevokizumab or canakinumab. In a further aspect provided herein is a patient's serum IL-6 level as a biomarker in the treatment of neuroinflammation, including AD, in a patient, wherein said patient is treated with canakinumab or a functional fragment thereof. In one aspect, the patient has a serum IL-6 level equal to or greater than about 2 pg/mL, equal to or greater than about 2.5 pg/mL, or equal to or greater than about 4 mg/L, before first administration of an IL-1β binding antibody (e.g., canakinumab or gevokizumab) or functional fragment thereof. In one aspect serum IL-6 level is determined by any known method wherein the serum IL-6 levels have been determined using a biological sample obtained from the patient.

In one aspect, provided herein is an IL-1β binding antibody (e.g., canakinumab or gevokizumab) or a functional fragment thereof, for use in a patient in need thereof in the treatment of a neuroinflammatory disorder, including AD.

Each and every embodiment provided in this application applies, separately or in combination, to these aspects.

### DETAILED DESCRIPTION

In one aspect, provided herein is the use of an IL-1β binding antibody (e.g., canakinumab or gevokizumab) or a functional fragment thereof, for the treatment a neuroinflammatory disorder, including AD.

Accordingly, provided herein are methods or uses for treating a neuroinflammatory disorder, including AD using an IL-1β binding antibody or a functional fragment thereof, wherein such IL-1β binding antibodies or functional fragments thereof can reduce inflammation, e.g., can inhibit IL-1β mediated inflammation.

As used herein, the term “neuroinflammatory disease or disorder” refers to a medical condition that is manifested by inflammation of the nervous tissue. It may be initiated in response to a variety of cues including infections, traumatic brain injury, toxic metabolites, or autoimmunity. Examples of neuroinflammatory diseases or disorders include, but are not limited to, Parkinson's Disease, Amyotrophic Lateral Sclerosis (ALS), Multiple Sclerosis (MS), Alzheimer's Disease (AD), Traumatic Brain Injury (TBI), irritable bowel syndrome, schizophrenia, bipolar disorder, depression, anxiety (e.g., generalized anxiety disorder, obsessive-compulsive disorder, and/or post-traumatic stress disorder), dementia, autism spectrum disorder (e.g., autism, asperger's disorder, pervasive developmental disorder, and/or childhood disintegrative disorder), ataxia telangiectasia, Cockayne syndrome, corticobasal degeneration, Creutzfeldt-Jakob disease, spinocerebellare ataxia type 3, neuroborreliosis, primary lateral sclerosis, progressive supranuclear palsy, Schilder disease, subacute combined degeneration of spinal cord secondary to pernicious anemia, drug-induced demyelination, radiation induced demyelination, spinal muscular atrophy, Tabes dorsales, spinal cord injury, chronic inflammatory demyelinating neuropathy, a congenital metabolic disorder, polymyositis, temporal arteritis, vasculitis, autism, and interstitial cystitis, Hurler-ScheieSyndrome, Hunter Syndrome, Sanfillipo Syndrome, Maroteaux-Lany Syndrome, Sly Syndrome, Fucosidosis, Alpha-mannosidosis, Beta-mannosidosis, Schindler Disease, Pompe Disease, and Infantile Neuronal Ceroid Lipofuscinosis.

As used herein, “early AD” refers to stages of sporadic Alzheimer's disease (AD) that occur before the onset of overt dementia and with MMSE>20. As used herein, early AD also includes prodromal AD or presymptomatic AD.

As used herein “early AD” also refers to mild AD or mild cognitive impairment (MCI) due to AD.

As used herein, the maximum MMSE score is 30 points. As used herein, a score of 20 to 24 suggests mild dementia, 13 to 20 suggests moderate dementia, and less than 12 indicates severe dementia. On average, the MMSE score of a person with Alzheimer's declines about two to four points each year.

As used herein, mild cognitive impairment (MCI) is a neurocognitive disorder which involves cognitive impairments beyond those expected based on an individual's age and education but which are not significant enough to interfere with instrumental activities of daily living. MCI may occur as a transitional stage between normal aging and dementia, especially Alzheimer's disease. It includes both memory and non-memory impairments. Mild cognitive impairment has been relisted as mild neurocognitive disorder in DSM-5, and in ICD-11.

As used herein, stage 3 MCI patients have characteristic pathophysiologic changes of AD, subtle or more apparent detectable abnormalities on sensitive neuropsychological measures, and mild but detectable functional impairment. The functional impairment in this stage is not severe enough to warrant a diagnosis of overt dementia.

As used herein, stage 4 MCI patients have mild dementia, substantial progressive cognitive impairment affecting several domains, and/or neurobehavioral disturbance, as documented by the individual's report or by observer (e.g., study partner) report or by change on longitudinal cognitive testing.

Clearly evident functional impact on daily life, affecting mainly instrumental activities, no longer fully independent/requires occasional assistance with daily life activities.

A diagnosis of probable MCI due to AD or mild AD according to the National Institute on Aging and the Alzheimer's Association (NIA-AA) criteria at screening and at least a 6 month decline in cognitive function prior to screening documented in the medical record.

In one embodiment, in any of the methods or uses provided herein, peripheral inflammation is associated with the neurodegenerative disorder, including AD, e.g., early AD. Such peripheral inflammation may be determined by any known method. In one embodiment, in any of the methods or uses described herein, peripheral inflammation is characterized in that serum IL-6 level is greater than about 2 pg/mL before first administration of said IL-1β binding antibody or functional fragment thereof. In another embodiment, serum IL-6 level is greater than about 2.5 pg/mL, or equal to or greater than about 4 pg/mL.

In one embodiment, in any of the methods or uses provided herein, AD is confirmed by amyloid and tau positivity, in particular by Aβ associated pathologic state (A₄₂, or Aβ₄₂/Aβ₄₀ ratio) and tau associated pathologic state (phosphorylated tau or total tau), before treatment according to the methods and uses provided herein. In a specific embodiment, AD is confirmed by positivity of (A) Aβ₁₋₄₂ (<599 pg/mL) or Aβ₁₋₄₂/Aβ₁₋₄₀ ratio (<0.069). In addition, in a further embodiment, an AD patient to be treated herein shall have greater than the lower 10th percentile in one of the following Tau markers (T): phospho tau (P-Tau) (>21.5 pg/mL) or (N) total tau (T-tau) (>146 pg/mL) to further classify the patients using the ATN profile.

As used herein, examples of IL-1β inhibitors include, but are not limited to, canakinumab or a functional fragment thereof, gevokizumab or a functional fragment thereof, Anakinra or a functional fragment thereof, diacerein, Rilonacept or a functional fragment thereof, IL-1 Affibody (SOBI 006, Z-FC (Swedish Orphan Biovitrum/Affibody)) or a functional fragment thereof, Lutikizumab (ABT-981) (Abbott) or a functional fragment thereof, CDP-484 (Celltech) or a functional fragment thereof, LY-2189102 (Eli Lilly and Co.), or a functional fragment thereof.

In one embodiment of any use or method described herein, said IL-1β binding antibody is canakinumab. Canakinumab (ACZ885 or Ilaris®) is a high-affinity, fully human monoclonal antibody of the IgG1/k to interleukin-1(3, developed for the treatment of IL-1β driven inflammatory diseases. It is designed to bind to human IL-1β and thus blocks the interaction of this cytokine with its receptors. Canakinumab is described in WO02/16436 (U.S. application Ser. No. 10/362,082) which is hereby incorporated by reference in its entirety, and described in WHO Drug Information RL 59, page 47, see also SEQ ID N^{o} 9 (light chin) and SEQ ID N^{o} 10 (heavy chain).

In other embodiments of any use or method described herein, said IL-1β binding antibody is gevokizumab. Gevokizumab (XOMA-052) is a high-affinity, humanized monoclonal antibody of the IgG2 isotype to interleukin-1(3, developed for the treatment of IL-1β driven inflammatory diseases. Gevokizumab modulates IL-1β binding to its signaling receptor. Gevokizumab is described in WO2007/002261 (U.S. application Ser. No. 11/472,813) which is hereby incorporated by reference in its entirety and in WHO Drug Information, RL 66, pages 314-315.

In one embodiment said IL-1β binding antibody is LY-2189102, which is a humanized interleukin-1 beta (IL-1β) monoclonal antibody.

In one embodiment said IL-1β binding antibody or a functional fragment thereof is CDP-484 (Celltech), which is an antibody fragment blocking IL-1β.

In one embodiment said IL-1β binding antibody or a functional fragment thereof is IL-1 Affibody (SOBI 006, Z-FC (Swedish Orphan Biovitrum/Affibody)).

Thus in one embodiment, the use or method described herein comprises administering the IL-1β binding antibody or a functional fragment thereof to a patient with a neuroinflammatory disorder, including AD, in the range of about 30 mg to about 750 mg per treatment, alternatively in the range of about 60 mg to about 400 mg per treatment, alternatively about 100 mg to about 600 mg, about 100 mg to about 450 mg, about 100 mg to about 300 mg, alternatively about 150 mg to about 600 mg, about 150 mg to about 450 mg, about 150 mg to about 300 mg, alternatively about 150 mg to about 300 mg per treatment; alternatively about 90 mg to about 300 mg, or about 90 mg to about 200 mg per treatment, alternatively at least about 150 mg, at least about 180 mg, at least about 300 mg, at least about 250 mg, at least about 300 mg per treatment. In one embodiment, the use or method described herein comprises administering the IL-1β binding antibody or a functional fragment thereof to a patient with a neuroinflammatory disorder, including AD, in the range of about 150 mg to 300 mg per treatment. In one embodiment the patient with a neuroinflammatory disorder, including AD, receives each treatment about every 2 weeks, about every three weeks, about every four weeks (monthly), about every 6 weeks, bimonthly (every 2 months) or quarterly (every 3 months). The term “per treatment”, as used in this application and particularly in this context, should be understood as the total amount of drug received per hospital visit or per self administration or per administration helped by a health care giver. In one embodiment, the total amount of drug received per treatment is administered to a patient within one day. In a specific embodiment, the drug received per treatment is administered within half a day. In a specific embodiment, the drug received per treatment is administered within 4 hours. In a specific embodiment, the drug received per treatment is administered within 2 hours.

In one embodiment, the patient with a neuroinflammatory disorder, including AD, receives a dose of about 90 mg to about 450 mg of the IL-1β binding antibody or a functional fragment thereof per treatment. In one embodiment the patient with a neuroinflammatory disorder, including AD receives an IL-1β binding antibody (e.g., canakinumab or gevokizumab) or a functional fragment thereof monthly. In one embodiment the patient with a neuroinflammatory disorder, including AD receives an IL-1β binding antibody (e.g., canakinumab or gevokizumab) or a functional fragment thereof about every three weeks. In one embodiment said patient receives an IL-1β binding antibody (e.g., canakinumab or gevokizumab) or a functional fragment thereof monthly. In one embodiment said patient receives an IL-1β binding antibody (e.g., canakinumab or gevokizumab) or a functional fragment thereof about every three weeks. In one embodiment the range of an IL-1β binding antibody (e.g., canakinumab or gevokizumab) or a functional fragment thereof is at least about 150 mg or at least about 200 mg. In one embodiment the range of an IL-1β binding antibody (e.g., canakinumab or gevokizumab) or a functional fragment thereof is about 180 mg to about 450 mg.

In one embodiment, any one of the described uses and methods comprises administering the IL-1β binding antibody (e.g., canakinumab or gevokizumab) or a functional fragment thereof to a patient with a neuroinflammatory disorder, including AD, in a total dose of from about 100 mg to about 750 mg, alternatively about 100 mg-600 mg, about 100 mg to about 450 mg, about 100 mg to about 300 mg, alternatively in a total dose of from about 150 mg-600 mg, about 150 mg to 450 mg, about 150 mg to 300 mg, alternatively in a total dose of at least about 150 mg, at least about 180 mg, at least about 250 mg, at least about 300 mg, over a period of about 2 weeks, about 3 weeks, about 4 weeks, about 6 weeks, about 8 weeks or about 12 weeks.

In one embodiment, any one of the described uses and methods comprises administering the IL-1β binding antibody (e.g., canakinumab or gevokizumab) or a functional fragment thereof to a patient with a neuroinflammatory disorder, including AD, in a total dose of from about 100 mg to about 750 mg, alternatively about 100 mg to about 600 mg, about 100 mg to about 450 mg, about 100 mg to about 300 mg, alternatively in a total dose of from about 150 mg to about 600 mg, about 150 mg to 450 mg, about 150 mg to 300 mg, alternatively in a total dose of at least about 150 mg, at least about 180 mg, at least about 250 mg, at least about 300 mg, over a period of about 4 weeks, In one embodiment total dose of an IL-1β binding antibody (e.g., canakinumab or gevokizumab) or a functional fragment thereof is about 180 mg to about 450 mg.

In one embodiment, the total dose of the IL-1β binding antibody (e.g., canakinumab or gevokizumab) or a functional fragment thereof is administered multiple times, e.g., 2, 3, or 4 times, over the above defined period. In one embodiment, the IL-1β binding antibody (e.g., canakinumab or gevokizumab) or a functional fragment thereof is administered once over the above defined period.

Sometimes it is desirable to quickly reduce inflammation of patients diagnosed with a neuroinflammatory disorder, e.g., AD. IL-1β auto-induction has been shown in human mononuclear blood, human vascular endothelial, and vascular smooth muscle cells in vitro and in rabbits in vivo where IL-1 has been shown to induce its own gene expression and circulating IL-1β level (Dinarello et al. 1987, Warner et al. 1987a, and Warner et al. 1987b).

Thus in one embodiment, the described uses and methods, while keeping the above described dosing schedules, additionally envisages a further administration of the IL-1β binding antibody (e.g., canakinumab or gevokizumab) or a functional fragment thereof within about two weeks (e.g., two weeks) apart from the first administration. Subsequently, a third and/or the further administrations are following the schedule of about every 2 weeks, about every 3 weeks, about every 4 weeks (monthly), about every 6 weeks, bimonthly (about every 2 months) or quarterly (about every 3 months).

In one embodiment, the IL-1β binding antibody is canakinumab, wherein canakinumab is administered to a patient with a neuroinflammatory disorder, including AD, in the range of about 100 mg to about 750 mg per treatment, alternatively about 100 mg to 600 mg, about 100 mg to about 450 mg, about 100 mg to about 300 mg, alternatively about 150 mg-about 600 mg, about 150 mg to about 450 mg, about 150 mg to about 300 mg per treatment, alternatively about 200 mg to about 400 mg, about 200 mg to about 300 mg, alternatively at least about 150 mg, at least about 200 mg, at least about 250 mg, at least about 300 mg per treatment. In one embodiment the patient with a neuroinflammatory disorder, e.g. AD, receives each treatment about every 2 weeks, about every 3 weeks, about every 4 weeks (about monthly), about every 6 weeks, bimonthly (about every 2 months) or quarterly (about every 3 months). In one embodiment the patient with a neuroinflammatory disorder, including AD receives canakinumab monthly or about every three weeks. In one embodiment, the dose range of canakinumab is about 150 mg to about 450 mg; and can be about 150 mg to 300 mg, or 300 mg to 450 mg per treatment. In one embodiment the dose range of canakinumab for patient with a neuroinflammatory disorder, e.g. AD, is about 200 mg to about 450 mg about every 3 weeks or monthly. In one embodiment, the dose of canakinumab for a patient with a neuroinflammatory disorder, including AD, is about 200 mg about every 3 weeks. In one embodiment, the dose of canakinumab for a patient with a neuroinflammatory disorder, e.g. AD, is about 200 mg monthly. In one embodiment, the patient with a neuroinflammatory disorder (e.g., AD) receives canakinumab monthly or about every three weeks. In one embodiment, the patient with a neuroinflammatory disorder, including AD receives canakinumab in the dose range of about 200 mg to about 450 mg monthly or about every three weeks. In one embodiment the patient with a neuroinflammatory disorder, including AD, receives canakinumab at a dose of about 200 mg monthly or about every three weeks. Under medical care, if a safety concern arises, the dose can be down-titrated. In some embodiments, the dose can be down-titrated by increasing the dosing interval, e.g., by doubling the dosing interval. For example about 200 mg monthly or about every 3 weeks regimen can be changed to every two month or about every 6 weeks respectively. In an alternative embodiment the patient with a neuroinflammatory disorder, including AD receives canakinumab at a dose of about 200 mg every two months or about every 6 weeks in the down-titration phase or in the maintenance phase independent from any safety issue or throughout the treatment phase.

In a further embodiment, canakinumab is administered at a dose of about 150 mg, followed by a second administration at a dose of about 150 mg at week 4 from the first administration, and by subsequent administration at a dose of 300 mg every four weeks, starting at week 4 from the second administration. In an alternative embodiment administered at a dose of about 300 mg about every four weeks (monthly).

The dose and dosing descriptions herein apply to the use and methods of use of canakinumab or a functional fragment of canakinumab described herein.

In one embodiment, any one of the described uses and methods comprises administering canakinumab to a patient with a neuroinflammatory disorder, including AD, in a total dose of from about 100 mg to about 750 mg, and can be about 100 mg-600 mg, 100 mg to 450 mg, 100 mg to 300 mg, alternatively 150 mg-600 mg, 150 mg to 450 mg, 150 mg to 300 mg, alternatively 150 mg to 300 mg, alternatively 300 mg to 450 mg; alternatively at least 150 mg, at least 200 mg, at least 250 mg, at least 300 mg, alternatively at least 300 mg, over a period of about 2 weeks, about 3 weeks, about 4 weeks, about 6 weeks, about 8 weeks or about 12 weeks. In a specific embodiment canakinumab is adminstered over a period of 4 weeks. In another specific embodiment In one embodiment, canakinumab is administered multiple times, e.g., 2, 3, or 4 times over the above defined period. In one embodiment, canakinumab is administered once over the above defined period. In one embodiment the total dose of canakinumab is about 200 mg to about 450 mg. In one embodiment the total dose of canakinumab is about 300 mg to about 450 mg. In one embodiment the total dose of canakinumab is about 350 mg to about 450 mg.

In one embodiment, any one of the described uses and methods, while keeping the above described dosing schedules, additionally envisages a second administration of canakinumab, wherein the second administration of canakinumab is about two weeks, e.g., two weeks apart from the first administration.

In one embodiment, any one of the described uses and methods comprises administering canakinumab at a dose of about 150 mg, about every 2 weeks, about every 3 weeks or about every four weeks (monthly).

In one embodiment, any one of the described uses and methods comprises administering canakinumab at a dose of about 300 mg about every 2 weeks, about every 3 weeks, monthly, about every 6 weeks, bimonthly (every 2 months) or quarterly (every 3 months).

In one embodiment, any one of the described uses and methods comprises administering canakinumab at a dose of 300 mg once per month (monthly). In a further embodiment, any one of the described uses and methods, while keeping the above described dosing schedules, additionally envisages a second administration of canakinumab at about 300 mg, wherein the second administration of canakinumab is at about two weeks apart from the first administration.

In one embodiment, any one of the described uses and methods comprises administering canakinumab at a dose of 300 mg once per month (monthly). In a further embodiment, any one of the described uses and methods, while keeping the above described dosing schedules, additionally envisages a second administration of canakinumab at 300 mg, wherein the second administration of canakinumab is two weeks apart from the first administration.

In one embodiment of any of the uses or methods of use described herein, canakinumab is administered to a patient in need at 300 mg twice over a two week period, followed by an administration 3 months after the second dose and then every 3 months.

In one embodiment, any one of the uses or methods of use described herein comprises administering gevokizumab to a patient with a neuroinflammatory disorder, including AD, in the range of about 30 mg to about 450 mg per treatment, alternatively 90 mg-450 mg, 90 mg to 360 mg, 90 mg to 270 mg, 90 mg to 180 mg per treatment; alternatively 120 mg-450 mg, 120 mg to 360 mg, 120 mg to 270 mg, 120 mg to 180 mg per treatment, alternatively 150 mg-450 mg, 150 mg to 360 mg, 150 mg to 270 mg, 150 mg to 180 mg per treatment, alternatively 180 mg-450 mg, 180 mg to 360 mg, 180 mg to 270 mg per treatment; alternatively about 60 mg to about 360 mg, about 60 mg to 180 mg per treatment; alternatively at least 150 mg, at least 180 mg, at least 240 mg, at least 270 mg per treatment. In one embodiment the patient with a neuroinflammatory disorder including AD, receives treatment every 2 weeks, every 3 weeks, monthly (every 4 weeks), every 6 weeks, bimonthly (every 2 months) or quarterly (every 3 months). In one embodiment the patient with a neuroinflammatory disorder, including AD, receives at least one, e.g., one treatment per month. In one embodiment the range of gevokizumab is 150 mg to 270 mg. In one embodiment the range of gevokizumab is 60 mg to 180 mg. In one embodiment the range of gevokizumab is 60 mg to 90 mg. In one embodiment the range of gevokizumab is 90 mg to 270 mg. In one embodiment the range of gevokizumab is 90 mg to 180 mg. In one embodiment the schedule is every 3 weeks or monthly. In one embodiment the patient receives gevokizumab 60 mg to 90 mg every 3 weeks. In one embodiment the patient receives gevokizumab 60 mg to 90 mg monthly. In one embodiment the patient receives gevokizumab about 90 mg to about 360 mg, 90 mg to about 270 mg, 120 mg to 270 mg, 90 mg to 180 mg, 120 mg to 180 mg, 120 mg or 90 mg every 3 weeks. In one embodiment the patient receives gevokizumab about 90 mg to about 360 mg, 90 mg to about 270 mg, 120 mg to 270 mg, 90 mg to 180 mg, 120 mg to 180 mg, 120 mg or 90 mg monthly.

In one embodiment the patient with a neuroinflammatory disorder, including AD, receives gevokizumab about 120 mg every 3 weeks. In one embodiment the patient receives gevokizumab about 120 mg monthly. In one embodiment the patient receives gevokizumab about 90 mg every 3 weeks. In one embodiment the patient receives gevokizumab about 90 mg monthly. In one embodiment the patient receives gevokizumab about 180 mg every 3 weeks. In one embodiment the patient receives gevokizumab about 180 mg monthly. In one embodiment the patient receives gevokizumab about 200 mg every 3 weeks. In one embodiment the patient receives gevokizumab about 200 mg monthly.

Under medical care, if needed, the dose can be down-titrated. In some embodiments, the dose can be down-titrated by increasing the dosing interval, e.g., by doubling the dosing interval. For example, about 120 mg monthly or every 3 weeks regimen can be changed to every two month or every 6 weeks respectively. In an alternative embodiment the patient with a neuroinflammatory disorder, including AD, receives gevokizumab at a dose of about 120 mg every two month or every 6 weeks in the down-titration phase or in the maintenance phase independent from any safety issue or throughout the treatment phase.

In one embodiment, gevokizumab or a functional fragment thereof is administered parenterally. In one embodiment gevokizumab or a functional fragment thereof is administered intravenously. In one embodiment gevokizumab is administered subcutaneously.

In one embodiment, gevokizumab is administered 20-120 mg, alternatively about 30-about 60 mg, about 30-about 90 mg, or about 60-about 90 mg; administered intravenously, every 3 weeks. In one embodiment, gevokizumab or is administered about 20-about 120 mg, alternatively about 30-about 60 mg, about 30-about 90 mg, or about 60-about 90 mg; administered intravenously, every 4 weeks. In one embodiment, gevokizumab is administered about 30-about 180 mg, alternatively about 30-60 mg, about 30-about 90 mg, about 60-about 90 mg, or about 90-about 120 mg; administered subcutaneously, every 3 weeks. In one embodiment, gevokizumab is administered 30-180 mg, alternatively about 30-about 60 mg, about 30-about 90 mg, about 60-about 90 mg, about 90-about 120 mg, or about 120 mg-about 180 mg; administered subcutaneously, every 4 weeks.

The dose and dosing descriptions herein apply to the use and methods of use of gevokizumab or a functional fragment of gevokizumab described herein.

In one embodiment, any one of the uses or methods of use described herein comprises administering gevokizumab to a patient with a neuroinflammatory disorder, including AD, in a total dose of 90 mg-450 mg, 90 mg to 360 mg, 90 mg to 270 mg, 90 mg to 180 mg, alternatively 120 mg-450 mg, 120 mg to 360 mg, 120 mg to 270 mg, 120 mg to 180 mg, alternatively 150 mg-450 mg, 150 mg to 360 mg, 150 mg to 270 mg, 150 mg to 180 mg, alternatively 180 mg-450 mg, 180 mg to 360 mg, 180 mg to 270 mg, alternatively at least 90 mg, at least 120 mg, at least 150 mg, at least 180 mg over a period of 2 weeks, 3 weeks, 4 weeks, 6 weeks, 8 weeks, or 12 weeks. In one embodiment, any one of the uses or methods of use described herein comprises administering gevokizumab to a patient with a neuroinflammatory disorder, including AD, in a total dose of 90 mg-450 mg, 90 mg to 360 mg, 90 mg to 270 mg, 90 mg to 180 mg, alternatively 120 mg-450 mg, 120 mg to 360 mg, 120 mg to 270 mg, 120 mg to 180 mg, alternatively 150 mg-450 mg, 150 mg to 360 mg, 150 mg to 270 mg, 150 mg to 180 mg, alternatively 180 mg-450 mg, 180 mg to 360 mg, 180 mg to 270 mg, alternatively at least 90 mg, at least 120 mg, at least 150 mg, at least 180 mg over a period of 4 weeks. In one embodiment, gevokizumab is administered multiple times, e.g., 2, 3, or 4 times over the above defined period. In one embodiment, gevokizumab is administered once over the above defined period. In one embodiment the total dose of gevokizumab is 180 mg to 360 mg. In one embodiment, the patient receives gevokizumab at least once, e.g., one treatment per month.

In one embodiment, any one of the uses or methods of use described herein, while keeping the above described dosing schedules, especially envisages the second administration of gevokizumab is at most two weeks, e.g., two weeks apart from the first administration.

In one embodiment, any one of the uses or methods of use described herein comprises administering gevokizumab at a dose of 60 mg every 2 weeks, every 3 weeks or monthly.

In one embodiment, any one of the uses or methods of use described herein comprises administering gevokizumab at a dose of 90 mg every 2 weeks, every 3 weeks or monthly.

In one embodiment, any one of the uses or methods of use described herein comprises administering gevokizumab at a dose of 180 mg every 2 weeks, every 3 weeks (±3 days), monthly, every 6 weeks, bimonthly (every 2 months) or quarterly (every 3 months).

In one embodiment, any one of the uses or methods of use described herein comprises administering gevokizumab at a dose of 180 mg once per month (monthly). In one further embodiment, any one of the uses or methods of use described herein, while keeping the above described dosing schedules, envisages the second administration of gevokizumab at 180 mg is at most two weeks, e.g., two weeks apart from the first administration.

In one embodiment of any one of the uses or methods of use described herein, said IL-1β binding antibody or functional fragment thereof is an IL-1β binding antibody. In one embodiment of any one of the uses or methods of use described herein, said IL-1β binding antibody or functional fragment thereof is capable of inhibiting the binding of IL-1β to its receptor and has a K_{D} for binding to IL-1β of about 50 pM or less.

In other embodiments of any one of the uses or methods of use described herein, said IL-1β binding antibody is selected from the group consisting of:

a) an IL-1β binding antibody directed to an antigenic epitope of human IL-1β which includes the loop comprising the Glu64 residue of the mature IL-1β, wherein said IL-1β binding antibody is capable of inhibiting the binding of IL-1β to its receptor, and further wherein said IL-1β binding antibody has a K_{D} for binding to IL-1β of about 50 pM or less;

b) an IL-1β binding antibody that competes with the binding of an IL-1β binding antibody comprising a VH domain comprising SEQ ID NO:1 and a VL domain comprising SEQ ID NO:2;

c) an IL-1β binding antibody comprising the three CDRs of SEQ ID NO:3, SEQ ID NO:4, SEQ ID NO:5;

d) an anti-IL-1β binding antibody comprising the three CDRs of SEQ ID NO:6, SEQ ID NO:7, SEQ ID NO:8;

e) an anti-IL-1β binding antibody comprising the three CDRs of SEQ ID NO:3, SEQ ID NO:4, SEQ ID NO:5 and the three CDRs of SEQ ID NO:6, SEQ ID NO:7, SEQ ID NO:8;

f) an anti-IL-1β binding antibody comprising a VH domain comprising SEQ ID NO:1;

g) an anti-IL-1β binding antibody comprising a VL domain comprising SEQ ID NO:2;

h) an anti-IL-1β binding antibody comprising a VH domain comprising SEQ ID NO:1 and a VL domain comprising SEQ ID NO:2.

In one embodiment of any one of the uses or methods of use described herein, said IL-1β binding antibody or fragment thereof comprises the 3 CDRs of SEQ ID NO:1 are set forth in SEQ ID NO:3, 4, and 5 and wherein the 3 CDRs of SEQ ID NO:2 are set forth in SEQ ID NO:6, 7, and 8.

In other embodiments of any one of the uses or methods of use described herein, the IL-1β binding antibody comprises:

a) a VH having a first CDR having 0, 1 or 2 amino acid substitutions in comparison to the CDR set forth in SEQ ID NO:3, a second CDR having 0, 1 or 2 amino acid substitutions in comparison to the CDR set forth in SEQ ID NO:3, a third CDR having 0, 1 or 2 amino acid substitutions in comparison to the CDR set forth in SEQ ID NO:5; and

b) a VL having a first CDR having 0, 1 or 2 amino acid substitutions in comparison to the CDR set forth in SEQ ID NO:6, a second CDR having 0, 1 or 2 amino acid substitutions in comparison to the CDR set forth in SEQ ID NO:7, and a third CDR having 0, 1 or 2 amino acid substitutions in comparison to the CDR set forth in SEQ ID NO:8, wherein said antibody has a K_{D} for IL-1beta of 50 pM or less and wherein said antibody inhibits the binding of IL-1β to its receptor.

c) an antibody light chain having 0, 1 or 2 amino acid substitutions in comparison to the antibody light chain set forth in SEQ ID NO:9 and an antibody heavy chain having 0, 1 or 2 amino acid substitutions in comparison to the antibody heavy chain set forth in SEQ ID NO:10

Substituted amino acids are ideally conservative substitutions, and once substituted a skilled artisan could use an assay such as those described in WO02/16436 (U.S. application Ser. No. 10/362,082) incorporated by reference herein.

In one embodiment of any one of the uses or methods of use described herein, said IL-1β binding antibody is canakinumab. In other embodiments of any one of the uses or methods of use described herein, said IL-1β binding antibody or functional fragment thereof is selected from the group consisting of XOMA 052 or gevokizumab, LY-2189102 or AMG-108 or a function fragment thereof.

In some embodiments of any one of the uses or methods of use described herein, the antibody or functional fragment thereof binds to human IL-1β with a dissociation constant of about 50 pM or less. In some embodiments, the antibody or functional fragment thereof binds to human IL-I β with a dissociation constant of about 500 pM or less. In some embodiments, the IL-1β binding antibody or functional fragment thereof binds to human IL-I 13 with a dissociation constant of about 250 pM or less. In some embodiments, the IL-1β binding antibody or functional fragment thereof binds to human IL-1β with a dissociation constant of about 100 pM or less. In some embodiments of any of the methods described above, the IL-1β binding antibody or functional fragment thereof binds to human IL-1β with a dissociation constant of about 5 pM or less. In some embodiments, the IL-1β binding antibody or functional fragment thereof binds to human IL-1β with a dissociation constant of about 1 pM or less. In some embodiments, the IL-1β binding antibody or functional fragment thereof binds to human IL-1β with dissociation constant of about 0.3 pM or less.

In some embodiments of any and/or all of the methods described above, the IL-1β binding antibody or functional fragment thereof is a neutralizing antibody.

The canakinumab heavy chain variable region (VH) is set forth as SEQ ID NO:1 of the sequence listing. CDR1 of the VH of canakinumab is set forth as SEQ ID NO:3 of the sequence listing. CDR2 of the VH of canakinumab is set forth as SEQ ID NO:4 of the sequence listing. CDR3 of the VH of canakinumab is set forth as SEQ ID NO:5 of the sequence listing.

The canakinumab light chain variable region (VL) is set forth as SEQ ID NO:2 of the sequence listing. CDR1 of the VL of canakinumab is set forth as SEQ ID NO:6 of the sequence listing. CDR2 of the VL of canakinumab is set forth as SEQ ID NO:7 of the sequence listing. CDR3 of the VL of canakinumab is set forth as SEQ ID NO:8 of the sequence listing.

In some embodiments of any and/or all of the methods described above, the anti-IL-1β binding antibody or binding fragment thereof competes with the binding of an antibody having the light chain variable region of SEQ ID NO:1 and the heavy chain variable region of SEQ ID NO:2.

As used herein, canakinumab is defined under INN number 8836 and has the following sequence:

| Light chain (SEQ ID NO: 9): | |
|---|---|
| 1 | EIVLTQSPDF QSVTPKEKVT ITCRASQSIG SSLHWYQQKP DQSPKLLIKY ASQSFSGVPS |
| 61 | RFSGSGSGTD FTLTINSLEA EDAAAYYCHQ SSSLPFTFGP GTKVDIKRTV AAPSVFIFPP |
| 121 | SDEQLKSGTA SVVCLLNNFY PREAKVQWKV DNALQSGNSQ ESVTEQDSKD STYSLSSTLT |
| 181 | LSKADYEKHK VYACEVTHQG LSSPVTKSFN RGEC* |
| Heavy chain (SEQ ID NO: 10): | |
| 1 | QVQLVESGGG VVQPGRSLRL SCAASGFTFS VYGMNWVRQA PGKGLEWVAI IWYDGDNQYY |
| 61 | ADSVKGRFTI SRDNSKNTLY LQMNGLRAED TAVYYCARDL RTGPFDYWGQ GTLVTVSSAS |
| 121 | TKGPSVFPLA PSSKSTSGGT AALGCLVKDY FPEPVTVSWN SGALTSGVHT FPAVLQSSGL |
| 181 | YSLSSVVTVP SSSLGTQTYI CNVNHKPSNT KVDKRVEPKS CDKTHTCPPC PAPELLGGPS |
| 241 | VFLFPPKPKD TLMISRTPEV TCVVVDVSHE DPEVKFNWYV DGVEVHNAKT KPREEQYNST |
| 301 | YRVVSVLTVL HQDWLNGKEY KCKVSNKALP APIEKTISKA KGQPREPQVY TLPPSREEMT |
| 361 | KNQVSLTCLV KGFYPSDIAV EWESNGQPEN NYKTTPPVLD SDGSFFLYSK LTVDKSRWQQ |
| 421 | GNVFSCSVMH EALHNHYTQK SLSLSPGK* |

As used herein gevokizumab, which is defined under INN number 9310, has the following sequence

| Heavy chain | |
|---|---|
| QVQLQESGPG LVKPSQTLSL TCSFSGFSLS TSGMGVGWIR QPSGKGLEWL | 50 |
| AHIWWDGDES YNPSLKSRLT ISKDTSKNQV SLKITSVTAA DTAVYFCARN | 100 |
| RYDPPWFVDW GQGTLVTVSS ASTKGPSVFP LAPCSRSTSE STAALGCLVK | 150 |
| DYFPEPVTVS WNSGALTSGV HTFPAVLQSS GLYSLSSVVT VTSSNFGTQT | 200 |
| YTCNVDHKPS NTKVDKTVER KCCVECPPCP APPVAGPSVF LFPPKPKDTL | 250 |
| MISRTPEVTC VVVDVSHEDP EVQFNWYVDG MEVHNAKTKP REEQFNSTFR | 300 |
| VVSVLTVVHQ DWLNGKEYKC KVSNKGLPAP IEKTISKTKG QPREPQVYTL | 350 |
| PPSREEMTKN QVSLTCLVKG FYPSDIAVEW ESNGQPENNY KTTPPMLDSD | 400 |
| GSFFLYSKLT VDKSRWQQGN VFSCSVMHEA LHNHYTQKSL SLSPG | 445 |
| Light chain | |
| DIQMTQSTSS LSASVGDRVT ITCRASQDIS NYLSWYQQKP GKAVKLLIYY | 50 |
| TSKLHSGVPS RFSGSGSGTD YTLTISSLQQ EDFATYFCLQ GKMLPWTFGQ | 100 |
| GTKLEIKRTV AAPSVFIFPP SDEQLKSGTA SVVCLLNNFY PREAKVQWKV | 150 |
| DNALQSGNSQ ESVTEQDSKD STYSLSSTLT LSKADYEKHK VYACEVTHQG | 200 |
| LSSPVTKSFN RGEC | 214 |

In one embodiment, treatment success is determined by a positive result on the NTB total score Neuropsychological test battery (NTB). In a specific embodiment, the effect size is 0.5 and corresponds to a moderate treatment effect.

In another embodiment, treatment success is determined by Clinical dementia rating scale (CDR), Mini mental state examination (MMSE), Montreal cognitive assessment (MoCA), Repeatable battery for the assessment of neuropsychological status (RBANS), Alzheimer's disease assessment scale, cognition (ADAS-Cog).

In one embodiment; treatment success is measured with PET-TSPO. In a specific embodiment, a reduction of 25% (true mean) in microglial activation due to an anti-inflammatory agent provides a measurement of treatment success.

In some aspects, provided herein are:
1. A method of treating a neuroinflammatory disorder in a patient, comprising administering an IL-1β binding antibody or functional fragment thereof
2. A method according to aspect 1, wherein the neuroinflammatory disorder is selected form the group of Alzheimer's Disease (AD), Parkinson's Disease, Amyotrophic Lateral Sclerosis (ALS), Multiple Sclerosis (MS), progressive supranuclear palsy (PSP), Traumatic Brain Injury (TBI), irritable bowel syndrome, schizophrenia, bipolar disorder, depression, anxiety (e.g., generalized anxiety disorder, obsessive-compulsive disorder and post-traumatic stress disorder, dementia, autism spectrum disorder (e.g., autism, asperger's disorder, pervasive developmental disorder, childhood disintegrative disorder), ataxia telangiectasia, Cockayne syndrome, corticobasal degeneration, Creutzfeldt-Jakob disease, spinocerebellare ataxia type 3, neuroborreliosis, primary lateral sclerosis, Schilder's disease, subacute combined degeneration of spinal cord secondary to pernicious anemia, drug-induced demyelination, radiation induced demyelination, spinal muscular atrophy, Tabes dorsales, spinal cord injury, chronic inflammatory demyelinating neuropathy, a congenital metabolic disorder, polymyositis, temporal arteritis, vasculitis, autism, and interstitial cystitis, Hurler-Scheie Syndrome, Hunter Syndrome, Sanfillipo Syndrome, Maroteaux-Lany Syndrome, Sly Syndrome, Fucosidosis, Alpha-mannosidosis, Beta-mannosidosis, Schindler Disease, Pompe Disease, and Infantile Neuronal Ceroid Lipofuscinosis.
3. A method according to aspect 2, wherein the disorder is Alzheimer's disease.
4. The method according to anyone of aspects 1-3 wherein Alzheimer's disease is early AD.
5. The method according to aspect 1-4, wherein early AD is mild AD or mild cognitive impairment (MCI) due to AD.
6. The method according to anyone of the preceding aspects, wherein the disease is associated with peripheral inflammation.
7. The method according to anyone of the preceding aspects, wherein said peripheral inflammation is characterized in that serum IL-6 level is greater than about 2 pg/mL before first administration of said IL-1β binding antibody or functional fragment thereof
8. The method according to anyone of the preceding aspects, wherein the interleukin-6 (IL-6) level of said patient has reduced by at least 20% compared to baseline assessed at least about 3 months after first administration of the IL-1β binding antibody or functional fragment thereof
9. The method according to anyone of the preceding aspects, wherein said IL-1β binding antibody or functional fragment thereof is selected from the group consisting of:
   a) an IL-1β binding antibody directed ton antigenic epitope of human IL-1β which includes the loop comprising the Glu64 residue of the mature IL-1β, wherein said IL-1β binding antibody is capable of inhibiting the binding of IL-1β to its receptor, and further wherein said IL-1β binding antibody has a K_{D} for binding to IL-1β of about 50 pM or less;
   b) an IL-1β binding antibody that competes with the binding of an IL-1β binding antibody comprising a VH domain comprising SEQ ID NO:1 and a VL domain comprising SEQ ID NO:2;
   c) an anti-IL-1β binding antibody comprising the three CDRs of SEQ ID NO:3, SEQ ID NO:4, SEQ ID NO:5;
   d) an anti-IL-1β binding antibody comprising the three CDRs of SEQ ID NO:6, SEQ ID NO:7, SEQ ID NO:8;
   e) an anti-IL-1β binding antibody comprising the three CDRs of SEQ ID NO:3, SEQ ID NO:4, SEQ ID NO:5 and the three CDRs of SEQ ID NO:6, SEQ ID NO:7, SEQ ID NO:8;
   f) an anti-IL-1β binding antibody comprising a VH domain comprising SEQ ID NO:1;
   g) an anti-IL-1β binding antibody comprising a VL domain comprising SEQ ID NO:2;
   h) an anti-IL-1β binding antibody comprising a VH domain comprising SEQ ID NO:1 and a VL domain comprising SEQ ID NO:2.
   i) an anti-IL-1β binding antibody comprising a light chain comprising SEQ ID NO:9.
   j) an anti-IL-1β binding antibody comprising a heavy chain comprising SEQ ID NO:10.
10. The method according to anyone of the preceding aspects, wherein said IL-1β binding antibody or a functional fragment thereof is canakinumab.
11. The method according to anyone of the preceding aspects, wherein canakinumab is administered at a dose of about 150 mg or about 300 mg per treatment.
12. The method according to aspect 10 or 11, wherein canakinumab is administered every two weeks, every three weeks or every four weeks (monthly).
13. The method according to anyone of aspects 10-12, wherein canakinumab is administered subcutaneously.
14. The method according to any one of aspects 10-12, wherein canakinumab is administered intravenously.
15. The method according to anyone of aspects 10-14, wherein canakinumab is administered at a dose of about 150 mg, followed by a second administration at a dose of about 150 mg at week 4 from the first administration, and by subsequent administration at a dose of 300 mg every four weeks, starting at week 4 from the second administration.
16. An IL-1β binding antibody or a functional fragment thereof for use in the treatment of a neuroinflammatory disorder.
17. An IL-1β binding antibody or a functional fragment thereof for use according to aspect 16, wherein the neuroinflammatory disorder is selected form the group of Alzheimer's Disease (AD), Parkinson's Disease, Amyotrophic Lateral Sclerosis (ALS), Multiple Sclerosis (MS), progressive supranuclear palsy (PSP), Traumatic Brain Injury (TBI), irritable bowel syndrome, schizophrenia, bipolar disorder, depression, anxiety (generalized anxiety disorder, obsessive-compulsive disorder and post-traumatic stress disorder), dementia, autism spectrum disorder (autism, asperger's disorder, pervasive developmental disorder, childhood disintegrative disorder), ataxia telangiectasia, Cockayne syndrome, corticobasal degeneration, Creutzfeldt-Jakob disease, spinocerebellare ataxia type 3, neuroborreliosis, primary lateral sclerosis, Schilder's disease, subacute combined degeneration of spinal cord secondary to pernicious anemia, drug-induced demyelination, radiation induced demyelination, spinal muscular atrophy, Tabes dorsalis, spinal cord injury, chronic inflammatory demyelinating neuropathy, a congenital metabolic disorder, polymyositis, temporal arteritis, vasculitis, autism, and interstitial cystitis, Hurler-Scheie Syndrome, Hunter Syndrome, Sanfillipo Syndrome, Maroteaux-Lany Syndrome, Sly Syndrome, Fucosidosis, Alpha-mannosidosis, Beta-mannosidosis, Schindler's Disease, Pompe Disease and Infantile Neuronal Ceroid Lipofuscinosis.
18. The IL-1β binding antibody or a functional fragment thereof for use according to aspect 16 or 17, wherein the disorder is Alzheimer's disease.
19. The IL-1β binding antibody or a functional fragment thereof for use according to anyone of aspects 16-18, wherein the Alzheimer's disease is early AD.
20. The IL-1β binding antibody or a functional fragment thereof for use according to one of aspects 16-19, wherein the early AD is mild AD or mild cognitive impairment (MCI) due to AD.
21. The IL-1β binding antibody or a functional fragment thereof for use according to anyone of aspects 16-20, wherein the disorder is associated with peripheral inflammation.
22. The IL-1β binding antibody or a functional fragment thereof for use according to anyone of aspects 16-21, wherein said peripheral inflammation is characterized in that serum IL-6 is greater than about 2 pg/mL before the first administration of said IL-1β binding antibody or functional fragment thereof
23. The IL-1β binding antibody or a functional fragment thereof for use according to any one of aspects 16-22, wherein the interleukin-6 (IL-6) level of said patient has decreased by at least 20% compared to baseline assessed at least about 3 months after first administration of the IL-1β binding antibody or functional fragment thereof
24. The IL-1β binding antibody or a functional fragment thereof for use according to anyone of aspects 16-23, wherein said IL-1β binding antibody or functional fragment thereof is selected from the group consisting of:
   a) an IL-1β binding antibody directed ton antigenic epitope of human IL-1β which includes the loop comprising the Glu64 residue of the mature IL-1β, wherein said IL-1β binding antibody is capable of inhibiting the binding of IL-1β to its receptor, and further wherein said IL-1β binding antibody has a K_{D} for binding to IL-1β of about 50 pM or less;
   b) an IL-1β binding antibody that competes with the binding of an IL-1β binding antibody comprising a VH domain comprising SEQ ID NO:1 and a VL domain comprising SEQ ID NO:2;
   c) an anti-IL-1β binding antibody comprising the three CDRs of SEQ ID NO:3, SEQ ID NO:4, SEQ ID NO:5;
   d) an anti-IL-1β binding antibody comprising the three CDRs of SEQ ID NO:6, SEQ ID NO:7, SEQ ID NO:8;
   e) an anti-IL-1β binding antibody comprising the three CDRs of SEQ ID NO:3, SEQ ID NO:4, SEQ ID NO:5 and the three CDRs of SEQ ID NO:6, SEQ ID NO:7, SEQ ID NO:8;
   f) an anti-IL-1β binding antibody comprising a VH domain comprising SEQ ID NO:1;
   g) an anti-IL-1β binding antibody comprising a VL domain comprising SEQ ID NO:2;
   h) an anti-IL-1β binding antibody comprising a VH domain comprising SEQ ID NO:1 and a VL domain comprising SEQ ID NO:2.
   i) an anti-IL-1β binding antibody comprising a light chain comprising SEQ ID NO:9.
   j) an anti-IL-1β binding antibody comprising a heavy chain comprising SEQ ID NO:10.
25. The IL-1β binding antibody or a functional fragment thereof for use according to anyone of aspects 16-24, wherein said IL-1β binding antibody is canakinumab
26. The IL-1β binding antibody or a functional fragment thereof for use according to aspect 16-25, wherein canakinumab is administered at a dose of about 150 mg or about 300 mg per treatment.
27. The IL-1β binding antibody or a functional fragment thereof for use according to any one of aspects 16-26, wherein canakinumab is administered about every two weeks, about every three weeks or about every four weeks (monthly).
28. The IL-1β binding antibody or a functional fragment thereof for use according to any one of aspects 16-27, wherein canakinumab is administered subcutaneously.
29. The IL-1β binding antibody or a functional fragment thereof for use according to any one of aspects 16-27, wherein canakinumab is administered intravenously.
30. The IL-1β binding antibody or a functional fragment thereof for use according to anyone of aspects 16-29, wherein canakinumab is administered at a dose of about 150 mg, followed by a dose of about 150 mg at week 4 from the first administration and by a dose of about 300 mg every four weeks, starting at week 4 from the second administration.
31. Canakinumab for use in the treatment of Alzheimer's disease, wherein canakinumab is administered at a dose of about 150 mg or about 300 mg subcutaneously about every four weeks.
32. Canakinumab for use according to aspect 31, wherein canakinumab is administered at a dose of 300 mg subcutaneously about every four weeks.
33. Canakinumab for use in the treatment of Alzheimer's disease, wherein canakinumab is administered at a first dose of about 150 mg, followed by a second dose of about 150 mg at about week 4 from the first administration and by a dose of 300 mg about every four weeks, starting at week 4 from the second administration.
34. A pharmaceutical composition for treating a neuroinflammatory disorder, wherein an IL-1β binding antibody or functional fragment thereof is administered at a dose of about 150 mg to about 300 mg.
35. A pharmaceutical composition according to aspect 34, wherein the disorder is Alzheimer's disease.
36. A pharmaceutical composition according to aspect 34 or 35, wherein said IL-1β binding antibody or functional fragment thereof is canakinumab.
37. Use of an IL-1β binding antibody for the manufacture of a medicament for treating a neuroinflammatory disorder.
38. The use according to aspect 37, wherein the disorder is Alzheimer's disease.

Other features, objects, and advantages of any one of the uses or methods of use described herein will be apparent from the description and drawings, and from the claims.

The following Examples illustrate the uses and methods described herein; they are not, however, intended to limit the scope in any way.

### Example 1

The Example below is set forth to aid in the understanding of the herein described uses and methods of use but is not intended, and should not be construed, to limit its scope in any way.

Study Design

This is a randomized, placebo-controlled, participant- and investigator-blinded study in participants with MCI due to AD or mild AD who have evidence of peripheral inflammation.

Rationale for Study Population

This clinical trial includes amyloid and tau confirmed early AD participants (MCI due to AD; stage 3 and mild AD; stage 4) with evidence of peripheral inflammation (elevated IL-6 levels in serum). This is in order to target individuals with the greatest likelihood of showing symptomatic enhancements from an anti-inflammatory treatment intervention.

The rationale for intervening during the early stages of AD is partially based on biomarker and PET imaging studies demonstrating evidence of brain inflammation increasing as disease progresses. Neuroinflammation, as an early feature of AD, can already be detected in the MCI stage prior to the onset of dementia (Bradburn et al 2019; Nordengen et al 2019; Parbo et al 2017).

Moreover, researchers have proposed that previous anti-inflammatory trials may not have been intervening early enough in the disease and therefore may not have been able to demonstrate beneficial drug treatment effects (King et al 2019).

According to the FDA's guidance for industry “U.S. Food and Drug Administration Center for Drug Evaluation and 2018,” drug development in participants who are early in the disease is advantageous for several reasons, including “the development of characteristic pathophysiological changes that greatly precede the development of clinically evident findings and the slowly progressive course of AD.” Participation in this trial requires that participants have AD. A clinical diagnosis of AD is not considered sufficient to reliably identify individuals with AD pathology. Confirmation of CSF amyloid and tau is considered crucial to establishing a robust diagnosis of AD. Clinically diagnosed MCI, without any biochemical verification, is an especially heterogeneous condition, which may result from various brain disorders such as AD, depression, traumatic brain injury, the prodromal phase of frontotemporal dementia and dementia with Lewy bodies. In fact, recent findings suggest that a substantial portion of clinically diagnosed MCI and AD participants lack biomarker evidence of AD. Incorrect inclusion of non-AD participants may complicate interpretation of clinical drug trials of AD. It is therefore of upmost importance to pursue AD pathology biomarker confirmation in any disease-modifying or symptomatic treatment trials including individuals with MCI due to AD (Cummings, 2019).

Following the NIA-AA research framework toward a biological definition of AD which classifies biomarkers into Aβ plaques (A), fibrillar tau (T) and neurodegeneration or neuronal injury (N) creates a combined AT(N) profile, which classifies participants on a syndromal cognitive staging.

The AT(N) classification is confirmed using soluble biomarkers for all participants enrolled in the study.

Clinical Staging

MCI/Stage 3
Performance in the impaired/abnormal range on objective cognitive tests;
Evidence of decline from baseline, documented by the individual's report or by observer (e.g., study partner) report or by change on longitudinal cognitive testing or neurobehavioral assessments;
May be characterized by cognitive presentations that are not primarily amnestic;
Performs daily life activities independently, but cognitive difficulty may result in detectable but mild functional impact on the more complex activities of daily life, that is, may take more time or be less efficient but still can complete, either self-reported or corroborated by a study partner.

Mild AD/Stage 4:
Mild dementia;
Substantial progressive cognitive impairment affecting several domains, and/or neurobehavioral disturbance. Documented by the individual's report or by observer (e.g., study partner) report or by change on longitudinal cognitive testing;
Clearly evident functional impact on daily life, affecting mainly instrumental activities. No longer fully independent/requires occasional assistance with daily life activities (Jack et al 2018).

Biomarker Staging:
CSF evidence of Aβ associated pathologic state (A₄₂, or Aβ₄₂/Aβ₄₀ ratio) and tau associated pathologic state (phosphorylated tau or total tau) (Jack et al 2018).

A key aspect of this study is to identify AD participants that are most likely to benefit from anti-inflammatory treatment. In addition to following the AT(N) framework, this trial only includes AD participants who show evidence of peripheral inflammation (Cummings, 2019). Clinical drug trials in non-AD indications have used inflammatory biomarkers to identify participants more likely to respond to anti-inflammatory agents (e.g. hsCRP in CANTOS trial on Canakinumab (Aday and Ridker 2018)).

It has been proposed that using soluble inflammatory markers may also help identify AD participants who are more likely to respond to anti-inflammatory treatment (Cummings, 2019). An inflammatory biomarker, IL-6, has been shown to be elevated in aging, in disease states, and in AD, which is used to screen for inflammatory status in the present study.

Rationale for Objectives (Endpoints)

Primary Endpoint

The success of each individual agent tested in the present clinical study is determined by a positive result on the NTB total score, the primary endpoint of this study. The NTB is a widely accepted and validated test battery that has been used in numerous industry-sponsored clinical drug trials in AD.

The NTB was selected as the primary endpoint of the present trial primarily for the three reasons detailed below:

Firstly, the NTB allows examination of pro-cognitive agent effects using a well-established neuropsychological assessment with psychometric properties suitable for the early stages of AD (Harrison et al 2007). In comparison, the psychometric limitations of the Alzheimer's Disease Assessment Scale—Cognitive subscale (ADAS-Cog) in participants with early AD are well-recognized (Karin et al 2014).

Secondly, the NTB is a composite of multiple globally-established neuropsychological tests that provide a thorough assessment of the cognitive domains affected by early AD, in particular, memory, executive function, attention and verbal fluency. In comparison, the ADAS-Cog and the Repeatable Battery for the Assessment of Neuropsychological Status (RBANS) lack comprehensive measures of executive function (Harrison et al 2007; Garcia et al 2008). Previous studies suggest executive function is the cognitive domain most strongly impacted by inflammation. Two studies showed that low-grade inflammation (e.g. IL-6, IL-8, TNF-α and CRP), was negatively associated with processing speed, attention and executive function, but not memory in older adults (Heringa et al 2014; Tegeler et al 2016).

Thirdly, the NTB has shown good assay sensitivity to symptomatic treatment effects in AD drug intervention trials where other endpoints have failed (Karin et al 2014; Gilman et al 2005). The NTB sub-tests selected for this trial include the Rey Auditory Verbal Learning Test—immediate and delayed recall (RAVLT-I and RAVLT-D), Wechsler Memory Scale—Digit Span (WMDS), Controlled Word Association Test (COWAT) and Category Fluency Test (CFT).

The NTB is administered directly to the participant by a trained test administrator. The total administration time is estimated to take 35 min on average. Scores of each of the individual sub-tests in the NTB are standardized into z-scores and then added up to provide an overall total score for the NTB.

For the primary endpoint, change in NTB total z-score at week 24, data from 36 evaluable participants per arm (investigational agent and placebo) provides ˜80% power to detect a statistically significant difference between the two groups at a 1-sided α=0.10 when the true standardized mean difference is 0.5. The effect size of 0.5 corresponds a moderate treatment effect which is thought to represent a clinically meaningful improvement at 24 weeks. To account for 15% dropout rate, approximately n=86 participants randomized in a 1:1 ratio would be needed to address the primary objective. These calculations take into account results of the published study by Frolich et al. (2019), where the standard deviation of the change in NTB total z-score was estimated to be ˜0.38 for both active and placebo groups.

For smaller true effect sizes, the power is lower (Table 1 below).

TABLE 1
Sensitivity of power to changes in assumptions

| SD (common | True mean | True effect | alpha | |
|---|---|---|---|---|
| to 2 arms) | difference (Δ) | size (Δ/SD) | (1-sided) | Power |
| 0.38 | 0.19 | 0.5 | 10% | 80% |
| 0.38 | 0.152 | 0.4 | 10% | 66% |
| 0.38 | 0.114 | 0.3 | 10% | 50% |
| 0.38 | 0.095 | 0.25 | 10% | 41% |

n = 86 participants are randomized in a 1:1 ratio and the dropout rate is 15% (thus we have 72 evaluable participants, 36 per arm).

Secondary Endpoint

PET TSPO is examined in a subset of participants (˜40% of randomly assigned participants). The PET TSPO signal is considered a marker of central inflammation (a marker for activated microglia and astrocytes) and the signal strength has been shown to correlate with worsening clinical severity in participants with MCI or AD, measures of cognition and various clinical scores (Zou et al 2020; Kreisl et al 2016; Kreisl et al 2013) Sample size calculations suggest that PET TSPO imaging requires smaller sample sizes than other endpoints in this study, in order to be statistically powered to detect agent treatment effects

Changes in microglia activation is assessed in whole, regional and focal brain regions from baseline to completion of week 12. For each agent, a positive readout on PET TSPO is considered as proof of mechanism.

For the secondary endpoint, PET TSPO, a comparison between an investigational agent and placebo is based on data from approximately 10 participants per arm. Assuming inter-participant variability of 20%-25%, the power to detect statistically significant treatment difference (using 1-sided α=−0.05) is shown in Table 2 below. For the 25% true mean reduction in microglial activation due to an anti-inflammatory agent vs. placebo, there is at least 87% power (assuming coefficient of variation is 25% or less).

TABLE 2
Power for treatment comparison using
log-transformed PET TSPO at week 12

| | True mean | | |
|---|---|---|---|
| Coefficient | reduction | | |
| of variation | (active vs. | α | |
| (CV) | placebo) | (1-sided) | Power |
| 0.2 | 30% | 5% | 99% |
| 0.2 | 25% | 5% | 93% |
| 0.2 | 20% | 5% | 78% |
| 0.25 | 30% | 5% | 93% |
| 0.25 | 25% | 5% | 81% |
| 0.25 | 20% | 5% | 62% |

The assumed sample size is n = 20 (10 per arm)

Interleukin-1

IL-1β is mainly produced by mononuclear phagocytes in response to injury and infection, and plays a clinically significant role in the pathobiology of autoinflammatory syndromes (e.g., CAPS, TRAPS, HIDS/MKD, FMF; Still's disease including both SJIA and AOSD, and gout). It may also play a key role in other chronic inflammatory conditions such as type 2 diabetes mellitus (T2DM) and atherosclerotic cardiovascular disease.

A meta-analysis of the literature revealed that IL-1β is one of the key peripheral cytokines that is raised in AD (Swardfager et al 2010). Increased levels of IL-1β have additionally been detected in the brain tissue of AD patients (Cacabelos et al 1994), and IL-1β polymorphisms appear to increase the risk of AD (Di Bona et al 2008). IL-1β levels are already elevated early on in the MCI stage of AD and appear to remain elevated as the disease progresses (Forlenza et al 2009).

IL-1β is a major proinflammatory cytokine in the brain, playing an integral role in the orchestration of other proinflammatory cytokines, such as TNF-a and IL-6. Elevated levels of IL-1 have been implicated with increased APP production, beta amyloid plaque deposition and the steps leading up to hyperphosphorylation of tau (Kinney et al 2018; Quintanilla et al 2004). It has also been suggested that the increase of APP and amyloid burden results in a vicious circle of IL-1β production and microglia activation (Kinney et al 2018). Disrupting IL-1β may delay the onset of neurodegeneration (Basu et al 2004).

Canakinumab is expected to treat the signs and symptoms of inflammation and potentially the underlying structural damage of the disease. Several genes involved in glial clearance of misfolded proteins and inflammatory reactions have been associated with increased risk of AD (Heneka et al 2015).

Canakinumab (ACZ885 or Ilaris®)

Canakinumab is a high-affinity human monoclonal antihuman interleukin-1B antibody of the IgG1/k isotype, which is marketed and under ongoing development for the treatment of IL-1β driven inflammatory and oncologic diseases. By binding specifically to human IL-1β, canakinumab blocks the interaction of IL-1β with the IL-1R, leading to inhibition of its downstream targets, thereby preventing IL-1 β-induced gene activation and the production of inflammatory mediators.

ACZ885 Non-Clinical Data

Canakinumab potently inhibits the biological activity of human IL-1β by preventing its interaction with the IL-1 receptor. Its specificity is confined to human and marmoset IL-1β, and it does not cross-react with cynomolgus or rhesus monkey IL-1β. Canakinumab has demonstrated pharmacological activity on inflammatory processes induced by human IL-1 in rodents.

The cardiovascular safety of canakinumab was assessed. While specific safety pharmacology studies have not been conducted to date, no clinical signs of qualitative or quantitative electrocardiographic changes attributable to canakinumab administration were observed in the toxicology studies performed. In addition, no abnormal binding to cardiac or cardiovascular tissues was identified in any of the cross-reactivity studies using marmoset and human tissues. In vivo, pharmacokinetic and toxicokinetic investigations in marmosets and rhesus monkeys characterized canakinumab as a typical IgG-type antibody with low serum clearance and long terminal half-life (6.5 to 8 days in marmosets and 15 days in rhesus monkeys). A low volume of distribution indicated that the compound has only a limited potential for distribution into intact, healthy tissues.

Canakinumab was administered twice weekly intravenously (i.v.) for up to 26 weeks at dose levels up to 100 mg/kg and twice weekly s.c. at a dose level of 150 mg/kg for up to 13 weeks in marmoset. In marmosets, canakinumab was well-tolerated at all dose levels investigated administered i.v. or s.c. No treatment-related deaths or clinical signs were observed. There was no evidence of test article-related adverse effects, either in terms of in-life investigations or during the post-mortem examinations. In one single study, following s.c. administration of canakinumab, histopathology examination revealed a dose-related, minimal lymphoid hyperplasia in male animals, which was considered unlikely to be of toxicological or biological significance since there were no changes in immunophenotyping and the observation was not present in females, nor was it present in males and females following i.v. administration. Plasma concentrations that are well-tolerated in animals for 13 weeks of s.c. dosing are in excess of at least 125-fold (Cmax) and 200-fold (Cav) the serum concentrations in patients with CAPS treated with the recommended clinical dose of 150 mg s.c. given every 8 weeks.

Local tolerance to canakinumab after i.v. and s.c. administration was demonstrated in marmosets. Additionally, a single intra-articular administration of canakinumab at a dose level of 10 mg/kg into the knee joint of female marmosets was very well tolerated.

Canakinumab Dose Rationale

Typically, a very small portion of systemically administered biotherapeutic antibodies crosses the blood brain barrier to enter the CNS with an estimated 0.1-0.2% of circulating antibodies found in the brain at steady-state concentrations (Yu and Watts 2013). To maximize the CNS exposure and expected pharmacological effect in the central nervous system, the clinical study escalates the dose regimen up to 300 mg s.c. every 4 weeks as this represents the currently highest approved dosing of canakinumab. US and European Health Authorities approvals for this dose regimen include different indications like TRAPS, HIDS/MKD and FMF and Still's disease (AOSD and SJIA).

In Novartis study CACZ885D2201, NOMID and CINCA patients with a history of severe CNS inflammation were treated with canakinumab at 150 mg s.c. (or 2 mg/kg in patients ≤40 kg) or 300 mg (or 4 mg/kg) with escalation up to 600 mg (or 8 mg/kg) up to every 4 weeks as necessary. In this 24-month open label study, approximately 0.3% of canakinumab was detected in the CSF of the patients as well as increased levels of IL-1beta, which indicates that canakinumab binds and stabilizes its target in the central nervous system.

For the current study, AD patients with an age of greater than or equal to 45 years and less than or equal to 90 years are enrolled. An elderly population was well represented in previous Novartis study CACZ885M2301 (CANTOS). In this double-blind, placebo-controlled, event-driven trial of quarterly subcutaneous canakinumab, 10061 stable post-myocardial infarction patients with elevated hsCRP were randomized for the prevention of recurrent cardiovascular events. 37.5% of these patients, who were at cardiovascular risk were ≥65 years old, as were very elderly patients (9.2% were ≥75 years old). The safety profile in elderly patients in CANTOS, treated with 300 mg canakinumab s.c. every 12 weeks was consistent with that in the overall population. In general, elderly patients have a higher rate of AEs, but that was the same for patients on canakinumab and on placebo.

Study Treatment

The medication at 1×150 mg canakinumab (ACZ885) solution plus 1×1 mL placebo solution is administered subcutaneously once every 4 weeks for the first 2 doses, followed by 2×150 mg canakinumab (ACZ885) solution administered subcutaneously once every 4 weeks.

Elevated Levels of Interleukin 6 (IL-6) in Alzheimer's Disease

Chronic inflammatory processes have been implicated in the neuropathology AD. Components of chronic inflammatory processes in AD include microglia and astrocytes, the complement system, and various inflammatory mediators (including cytokines and chemokines). In addition, Aβ has been shown to induce expression of Interleukin 6 (IL-6) in astrocytes and microglia (Lai et al., 2017). IL-6 is an interleukin that acts as both a pro-inflammatory cytokine and an anti-inflammatory myokine. Induction of the inflammatory signaling pathways leads to the production and release of immune mediators leading to cell death, compromised neuronal function and increased blood-brain barrier permeability to leucocytes, neurotoxic cytokines and chemokines into brain (Ardura-Fabregat et al., 2017; Lai et al., 2017) Several studies have reported increased levels of IL-6 in patients with AD vs controls (Wu et al., 2015; Lee at al., 2009). The increased levels of IL-6 in patients with AD are significantly above the healthy control mean value of 1.46 pg/mL in data generated using Quantikine human IL-6 HS kit (R&D Systems) and within similar ranges previously published in aged controls (Yaffe et al., 2003; Lee et al., 2012). In addition, aging along with development of comorbidities has shown significant increase of IL-6 with a nearly ˜18% rate of increase which significantly increases with development of three or more diseases (Zhu et al., 2009). Finally, individuals with increased levels of IL-6 were more likely to experience a decline in global cognitive and executive functioning during a longitudinal evaluation (i.e., years) compared to those with lower IL-6 levels (Schram et al., 2007; Bradburn et al., 2018).

Patients with serum IL-6 levels greater than 2 pg/mL are treated with canakinumab under the present dosing regimen

In conclusion, IL-6 has been extensively investigated in both pre-clinical and clinical studies as a pro-inflammatory cytokine that can accelerate the neurodegenerative processes in AD. Increased levels of IL-6 have been found in patients with AD, individuals with significant comorbidities and has been associated with significant decline and cognitive and executive function. Increasing deposition of Aβ, a key pathological hallmark of AD, leads to microglia and astrocytes releasing IL-6 in brain. Thus, increased levels of IL-6 in a patient with AD may be useful as a marker to examine the status of chronic inflammation in that patient.

### REFERENCES

Aday A W, Ridker P M (2018) Antiinflammatory Therapy in Clinical Care: The CANTOS Trial and Beyond. Front Cardiovasc Med; 5:1-6.
Ardura-Fabregat A. et al. Targeting Neuroinflammation to Treat Alzheimer's Disease; CNS Drugs volume 31, pages 1057-1082(2017).
Basu A, Krady J K, Levison S W (2004) Interleukin-1: a master regulator of neuroinflammation. J. Neurosci. Res; 78:151-6.
Bradburn S, Sarginson J, Murgatroyd C A. Association of Peripheral Interleukin-6 with Global Cognitive Decline in Non-demented Adults: A Meta-Analysis of Prospective Studies. Front
Aging Neurosci. 2018 Jan. 8; 9:438 Bradburn S, Murgatroyd C, Ray N (2019) Neuroinflammation in mild cognitive impairment and Alzheimer's disease: A meta-analysis. Ageing Res. Rev; 50:1-8.
Cacabelos R, Alvarez X A, Fernández-Novoa L, et al (1994) Brain interleukin-1 beta in Alzheimer's disease and vascular dementia. Methods Find Exp Clin Pharmacol; 16:141-51.
Chou R C, Kane M, Ghimire S, et al (2016) Treatment for Rheumatoid Arthritis and Risk of Alzheimer's Disease: A Nested Case-Control Analysis. CNS Drugs; 30:1111-1120.
Cummings J (2019) The Role of Biomarkers in Alzheimer's Disease Drug Development. Adv. Exp. Med. Biol; 1118:29-61.
Di Bona D, Plaia A, Vasto S, et al (2008) Association between the interleukin-1beta polymorphisms and Alzheimer's disease: a systematic review and meta-analysis. Brain Res Rev; 59:155-63.
Forlenza O V, Diniz B S, Talib L L, et al (2009) Increased serum IL-1beta level in Alzheimer's disease and mild cognitive impairment. Dement Geriatr Cogn Disord; 28:507-12.
Garcia C, Leahy B, Corradi K, et al (2008) Component structure of the Repeatable Battery for the Assessment of Neuropsychological Status in dementia. Arch Clin Neuropsychol; 23:63-72.
Harrison J, Minassian S L, Jenkins L, et al (2007) A neuropsychological test battery for use in Alzheimer disease clinical trials. Arch. Neurol; 64:1323.
Gilman S, Koller M, Black R S, et al (2005) Clinical effects of Abeta immunization (AN1792) in patients with AD in an interrupted trial. Neurology; 64:1553-62.
Heringa S M, van den Berg E, Reijmer Y D, et al (2014) Markers of low-grade inflammation and endothelial dysfunction are related to reduced information processing speed and executive functioning in an older population—the Hoorn Study. Psychoneuroendocrinology; 40:108-18.
Heneka M T, Carson M J, El Khoury J, et al (2015) Neuroinflammation in Alzheimer's disease. Lancet Neurol; 14:388-405.
Hori Y, Takeda S, Cho H, et al (2015) A Food and Drug Administration-approved asthma therapeutic agent impacts amyloid (3 in the brain in a transgenic model of Alzheimer disease. J. Biol. Chem; 290:1966-78.
Jack C R, Bennett D A, Blennow K, et al (2018) NIA-AA Research Framework: Toward a biological definition of Alzheimer's disease. Alzheimers Dement; 14:535-562.
Kang Soo Lee, Ji Hyung Chung, Tae Kyou Choi, Shin Young Suh, Byoung Hoon Oh, Chang Hyung Hong Dement Geriatr Cogn Disord. 2009; 28(4):281-7, Peripheral cytokines and chemokines in Alzheimer's disease
Karin A, Hannesdottir K, Jaeger J, et al (2014) Psychometric evaluation of ADAS-Cog and NTB for measuring drug response. Acta Neurol. Scand; 129:114-22.
Kinney J W, Bemiller S M, Murtishaw A S, et al (2018) Inflammation as a central mechanism in Alzheimer's disease. Alzheimers Dement (N Y); 4:575-590. Kreisl W C, Lyoo C H, Liow J S, et al (2016) (11)C-PBR28 binding to translocator protein increases with progression of Alzheimer's disease. Neurobiol. Aging; 44:53-61.
Kreisl W C, Lyoo C H, McGwier M, et al (2013) In vivo radioligand binding to translocator protein correlates with severity of Alzheimer's disease. Brain; 136:2228-38.
Lai K. S. P., Liu C. S., Rau A., Lanctôt K. L., Köhler C. A., Pakosh M., et al. (2017). Peripheral inflammatory markers in Alzheimer's disease: a systematic review and meta-analysis of 175 studies. J. Neurol. Neurosurg. Psychiatry 88, 876-88.
Nordengen K, Kirsebom B E, Henjum K, et al (2019) Glial activation and inflammation along the Alzheimer's disease continuum. J Neuroinflammation; 16:46.
Parbo P, Ismail R, Hansen K V, et al (2017) Brain inflammation accompanies amyloid in the majority of mild cognitive impairment cases due to Alzheimer's disease. Brain; 140:2002-2011.
Perez-Nievas B G, Stein T D, Tai H C, et al (2013) Dissecting phenotypic traits linked to human resilience to Alzheimer's pathology. Brain; 136:2510-26.
Polsek D, Gildeh N, Cash D, et al (2018) Obstructive sleep apnoea and Alzheimer's disease: In search of shared pathomechanisms. Neurosci Biobehav Rev; 86:142-149.
Ridker, P. M., Everett, B. M., Thuren, T., MacFadyen, J. G., Chang, W. H., Ballantyne, C., Fonseca, F., Nicolau, J., Koenig, W., Anker, S. D. and Kastelein, J. J., 2017. Antiinflammatory therapy with canakinumab for atherosclerotic disease. New England Journal of Medicine, 377(12):1119-1131.
Schram M T, Euser S M, de Craen A J, Witteman J C, Frolich M, Hofman A, Jolles J, Breteler M M, Westendorp R G. Systemic markers of inflammation and cognitive decline in old age. J Am Geriatr Soc. 2007 May; 55(5):708-16.
Swardfager W, Lanctot K, Rothenburg L, et al (2010) A meta-analysis of cytokines in Alzheimer's disease. Biol. Psychiatry; 68:930-41.
Tao Q, Ang T F A, DeCarli C, et al (2018) Association of Chronic Low-grade Inflammation With Risk of Alzheimer Disease in ApoE4 Carriers. JAMA Netw Open; 1:e183597.
Tegeler C, O'Sullivan J L, Bucholtz N, et al (2016) The inflammatory markers CRP, IL-6, and IL-10 are associated with cognitive function—data from the Berlin Aging Study II. Neurobiol. Aging; 38:112-117.
Teixeira F B, Saito M T, Matheus F C, et al (2017) Periodontitis and Alzheimer's Disease: A Possible Comorbidity between Oral Chronic Inflammatory Condition and Neuroinflammation. Front Aging Neurosci; 10:327.
Tegeler C, O'Sullivan J L, Bucholtz N, et al (2016) The inflammatory markers CRP, IL-6, and IL-10 are associated with cognitive function—data from the Berlin Aging Study II. Neurobiol. Aging; 38:112-117.
U.S. Food and Drug Administration Center for Drug Evaluation and Research (2018) Early Alzheimer's Disease: Developing Drugs for Treatment. Guidance for Industry. Draft Guidance.
Wang H, Mei Zl, Zhong K L, et al (2014) Pretreatment with antiasthmatic drug ibudilast ameliorates Aβ 1-42-induced memory impairment and neurotoxicity in mice. Pharmacol. Biochem. Behav; 124:373-9.
Ya-Ying Wu, Jung-Lung Hsu, Han-Cheng Wang, Shyh-Jong Wu, Chen-Jee Hong, Irene Han-Juo Cheng Alterations of the Neuroinflammatory Markers IL-6 and TRAIL in Alzheimer's Disease Dement Geriatr Cogn Dis Extra 2015 Nov. 24; 5(3):424-34.
Yaffe K, Lindquist K, Penninx B W, Simonsick E M, Pahor M, Kritchevsky S, Launer L, Kuller L, Rubin S, Harris T, Neurology, Inflammatory markers and cognition in well-functioning African-American and white elders, 1 Jul. 2003, 61(1):76-80;
Yu Y J, Watts R J (2013) Developing therapeutic antibodies for neurodegenerative disease. Neurotherapeutics; 10:459-72.
Zhang C, Griciuc A, Hudry E, et al (2018) Cromolyn Reduces Levels of the Alzheimer's Disease-Associated Amyloid β-Protein by Promoting Microglial Phagocytosis. Sci Rep; 8:1144.
Zhu S, Patel K V, Bandinelli S, Ferrucci L, Guralnik J M. Predictors of interleukin-6 elevation in older adults. J Am Geriatr Soc. 2009 September; 57(9):1672-7.
Zou J, Tao S, Johnson A, et al (2020) Microglial activation, but not tau pathology, is independently associated with amyloid positivity and memory impairment. Neurobiol. Aging; 85:11-21

1. A method of treating a neuroinflammatory disorder in a patient, comprising administering an IL-1β binding antibody or functional fragment thereof.

2. A method according to claim 1, wherein the neuroinflammatory disorder is selected form the group of Alzheimer's Disease (AD), Parkinson's Disease, Amyotrophic Lateral Sclerosis (ALS), Multiple Sclerosis (MS), progressive supranuclear palsy (PSP), Traumatic Brain Injury (TBI), irritable bowel syndrome, schizophrenia, bipolar disorder, depression, anxiety (generalized anxiety disorder, obsessive-compulsive disorder and post-traumatic stress disorder, dementia, autism spectrum disorder (autism, asperger's disorder, pervasive developmental disorder, childhood disintegrative disorder), ataxia telangiectasia, Cockayne syndrome, corticobasal degeneration, Creutzfeldt-Jakob disease, spinocerebellare ataxia type 3, neuroborreliosis, primary lateral sclerosis, Schilder's disease, subacute combined degeneration of spinal cord secondary to pernicious anemia, drug-induced demyelination, radiation induced demyelination, spinal muscular atrophy, Tabes dorsales, spinal cord injury, chronic inflammatory demyelinating neuropathy, a congenital metabolic disorder, polymyositis, temporal arteritis, vasculitis, autism, and interstitial cystitis, Hurler-Scheie Syndrome, Hunter Syndrome, Sanfillipo Syndrome, Maroteaux-Lany Syndrome, Sly Syndrome, Fucosidosis, Alpha-mannosidosis, Beta-mannosidosis, Schindler's Disease, Pompe Disease, and Infantile Neuronal Ceroid Lipofuscinosis.

3. A method according to claim 2, wherein the disorder is Alzheimer's disease.

4. The method according to claim 3 wherein the Alzheimer's disease is early AD.

5. The method according to claim 4, wherein the early AD is mild AD or mild cognitive impairment (MCI) due to AD.

6. The method according to claim 1, wherein the disease is associated with peripheral inflammation.

7. The method according to claim 6, wherein said peripheral inflammation is characterized in that serum IL-6 level is greater than about 2 pg/mL before first administration of said IL-1β binding antibody or functional fragment thereof.

8. The method according to claim 7, wherein the interleukin-6 (IL-6) level of said patient has reduced by at least 20% compared to baseline assessed at least about 3 months after first administration of the IL-1β binding antibody or functional fragment thereof.

9. The method according to claim 1, wherein said IL-1β binding antibody or functional fragment thereof is selected from the group consisting of:
a) an IL-1β binding antibody directed ton antigenic epitope of human IL-1β which includes the loop comprising the Glu64 residue of the mature IL-1β, wherein said IL-1β binding antibody is capable of inhibiting the binding of IL-1β to its receptor, and further wherein said IL-1β binding antibody has a K_{D} for binding to IL-1β of about 50 pM or less;
b) an IL-1β binding antibody that competes with the binding of an IL-1β binding antibody comprising a VH domain comprising SEQ ID NO:1 and a VL domain comprising SEQ ID NO:2;
c) an anti-IL-1β binding antibody comprising the three CDRs of SEQ ID NO:3, SEQ ID NO:4, SEQ ID NO:5;
d) an anti-IL-Iβ binding antibody comprising the three CDRs of SEQ ID NO:6, SEQ ID NO:7, SEQ ID NO:8;
e) an anti-IL-Iβ binding antibody comprising the three CDRs of SEQ ID NO:3, SEQ ID NO:4, SEQ ID NO:5 and the three CDRs of SEQ ID NO:6, SEQ ID NO:7, SEQ ID NO:8;
f) an anti-IL-Iβ binding antibody comprising a VH domain comprising SEQ ID NO:1;
g) an anti-IL-Iβ binding antibody comprising a VL domain comprising SEQ ID NO:2;
h) an anti-IL-Iβ binding antibody comprising a VH domain comprising SEQ ID NO:1 and a VL domain comprising SEQ ID NO:2.
i) an anti-IL-Iβ binding antibody comprising a light chain comprising SEQ ID NO:9.
j) an anti-IL-Iβ binding antibody comprising a heavy chain comprising SEQ ID NO:10.

10. The method according to claim 1, wherein said IL-1β binding antibody or a functional fragment thereof is canakinumab.

11. The method according to claim 10, wherein canakinumab is administered at a dose of about 150 mg or about 300 mg per treatment.

12. The method according to claim 10, wherein canakinumab is administered every two weeks, every three weeks or every four weeks (monthly).

13. The method according to claim 10, wherein canakinumab is administered subcutaneously.

14. The method according to claim 10, wherein canakinumab is administered intravenously.

15. The method according to claim 10, wherein canakinumab is administered at a dose of about 150 mg, followed by a second administration at a dose of about 150 mg at week 4 from the first administration, and by subsequent administration at a dose of 300 mg every four weeks, starting at week 4 from the second administration.

16.-30. (canceled)

31. The method according to claim 10, wherein canakinumab is administered at a dose of 150 mg or 300 mg subcutaneously every four weeks.

32.-38. (canceled)


SEQUENCE LISTING (10 sequences)

SEQ ID NO: 1 - protein, 118 aa, Homo sapiens [sequence omitted]

SEQ ID NO: 2 - protein, 107 aa, Homo sapiens [sequence omitted]

SEQ ID NO: 3 - protein, 5 aa, Homo sapiens
VYGMN

SEQ ID NO: 4 - protein, 17 aa, Homo sapiens
IIWYDGDNQYYADSVKG

SEQ ID NO: 5 - protein, 6 aa, Homo sapiens
DLRTGP

SEQ ID NO: 6 - protein, 11 aa, Homo sapiens
RASQSIGSSLH

SEQ ID NO: 7 - protein, 6 aa, Homo sapiens
ASQSFS

SEQ ID NO: 8 - protein, 7 aa, Homo sapiens
HQSSSLP

SEQ ID NO: 9 - protein, 214 aa, Artificial Sequence
  Description of Artificial Sequence Synthetic polypeptide
EIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 448 aa, Artificial Sequence
  Description of Artificial Sequence Synthetic polypeptide
QVQLVESGGGVVQPGRSLRLSCAASGFTFSVYGMNWVRQAPGKGLEWVAIIWYDGDNQYYADSVKGRFTISRDNSKNTLYLQMNGLRAEDTAVYYCARDLRTGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK